(19) United States
(12) Patent Application Publication
(10) Pub. No.: US 2009/0043115 A1
(43) Pub. Date: Feb. 12, 2009

(54) Process for Producing Simvastatin

(75) Inventors: Hongping Ye, Anhui (CN); Meg M. Sun, San Diego, CA (US); Zuolin Zhu, San Diego, CA (US)

Correspondence Address: HAMRE, SCHUMANN, MUELLER & LARSON, P.C., P.O. BOX 2902, MINNEAPOLIS, MN, 55402-0902, US

(73) Assignee: PFICKER PHARMACEUTICALS LTD., Huaibei (CN)

(21) Appl. No.: 11/576,424

(22) PCT Filed: Sep. 26, 2005
(86) PCT No.: PCT/CN05/01572

§ 371 Date: Feb. 22, 2008

(30) Foreign Application Priority Data
Sep. 30, 2004 (CN) 200410084820.X

(51) Int. Cl. C07D 309/30; C07C 69/76; C07C 63/33; C07D 319/06

(52) U.S. Cl. 549/291; 560/102; 562/492; 549/374

(57) ABSTRACT

The present invention discloses a process for producing Simvastatin and intermediate thereof. The present invention uses inexpensive and easily available reagents, its condition is mild, and it leaves out the protective and deprotective steps, which are necessary in prior methods. Compared with prior art, the esterifying condition in 8-position is greatly simplified.

### FIELD OF THE INVENTION

The invention relates to methods for preparing simvastatin and intermediates thereof.

### BACKGROUND OF THE INVENTION

Simvastatin, is, [(1S,3R,7R,8S,8αR)-8-[2-[(2R,4R)-4-hydroxy-6-oxo-oxan-2-yl]ethyl]-3,7-dimethyl-1,2,3,7,8,8α-hexahydronaphthalen-1-yl]2,2-dimethylbutanoate, with a molecular structure shown as in a formula (1):

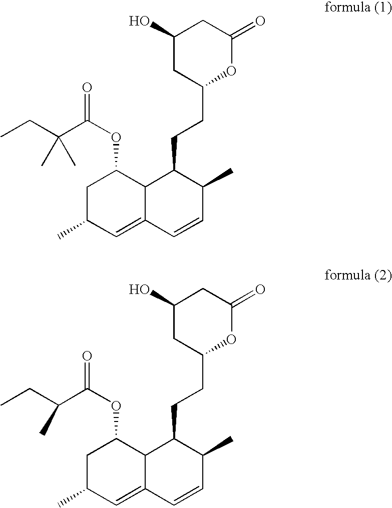

Tolerability of simvastatin is generally good, and most of adverse reactions associated with simvastatin are mild, for instance, less than 2% of patients dropped out from clinical trials due to adverse reactions. In 2002, the sales of simvastatin in North America is more than $5.7 billion USD.

Simvastatin is an HMG-CoA reductase inhibitor, which is prepared from lovastatin (as shown in the formula (2)) via semi-synthesis. The only difference between them is that they have a different functional group on 8-position: for lovastatin, it is 2-methyl butyryl; and for simvastatin, it is 2,2-dimethyl butyryl.

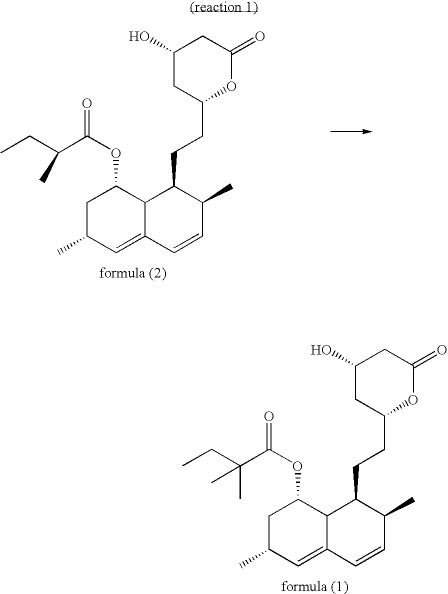

At present, known methods for producing simvastatin mainly includes the following two types:

The first manufacturing process is illustrated bellow. The process includes thoroughly removing the 2-methyl butyryl group at the 8-position of lovastatin, then replacing it with the 2,2-dimethyl butyryl group, therefore forming simvastatin. A number of patents disclosed this kind of process and the improvements thereof, for example, Canada patent No. 1,199,322, U.S. Pat. No. 5,159,104; U.S. Pat. No. 4,450,171; U.S. Pat. No. 4,444,784; U.S. Pat. No. 6,506,929; and U.S. Pat. No. 6,384,238. Disadvantages of these processes are the necessity of using TBDMS, which has to use very expensive raw material (TBDMSCl) as protective group for hydroxyl group(s), and/or the necessity of using more than eight times of 2,2-dimethyl butyryl chloride (which is a starting material) as an acylating agent, which means the reaction should be carried out in the presence of anhydrous pyridine, and the reaction time is excessively long, resulting difficulties in controlling reaction conditions, meanwhile, the excessively long reaction time also leads to difficult purification due to too much byproducts derived from elimination reactions in final product.

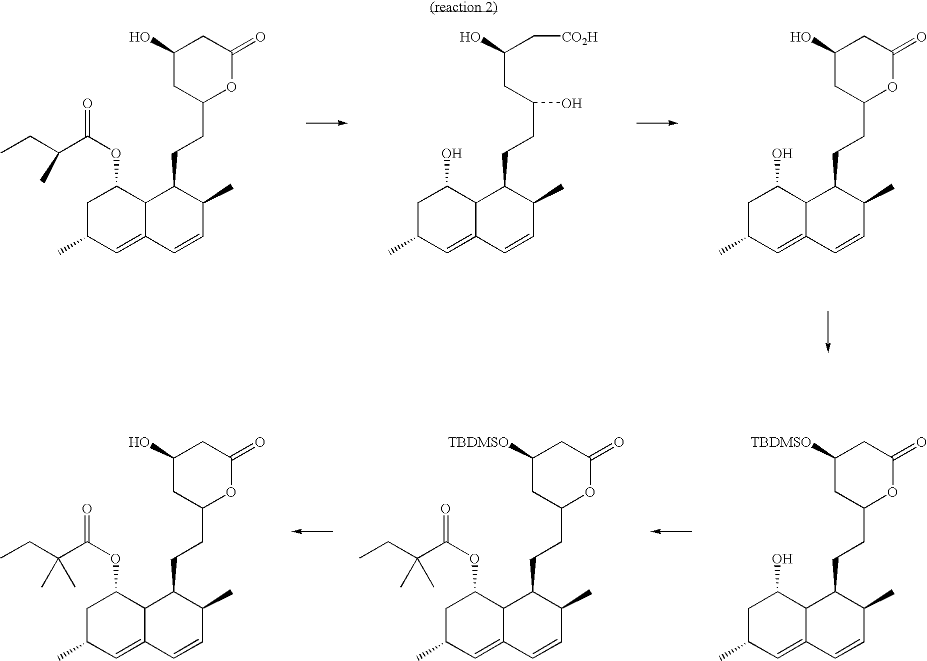

The second preparation method is illustrated bellow. The characteristics of the process is that it does not require removing 2-methyl-butyryl group in the 8-position of lovastatin, but adding a methyl group to the 2-methyl-butyryl group in the 8-position by protecting all other groups as shown. Also many patents disclose such preparation method(s) and various improvements thereof, for example, Canada patent No. 1,287,063, U.S. Pat. No. 5,393,983, U.S. Pat. No.4,582,915, U.S. Pat. No. 5, 763,646, U.S. Pat. No. 5,763,653, U.S. Pat. No. 6,100,407 and U.S. Pat. No. 6,384,238. Shortcomings of such a preparation method are that too many steps are included, expensive reagents are used, moreover, such methylation step is carried out under a temperature of less than −50° C., which means special equipments are required, energy consumption is too much, and the yields are too low.

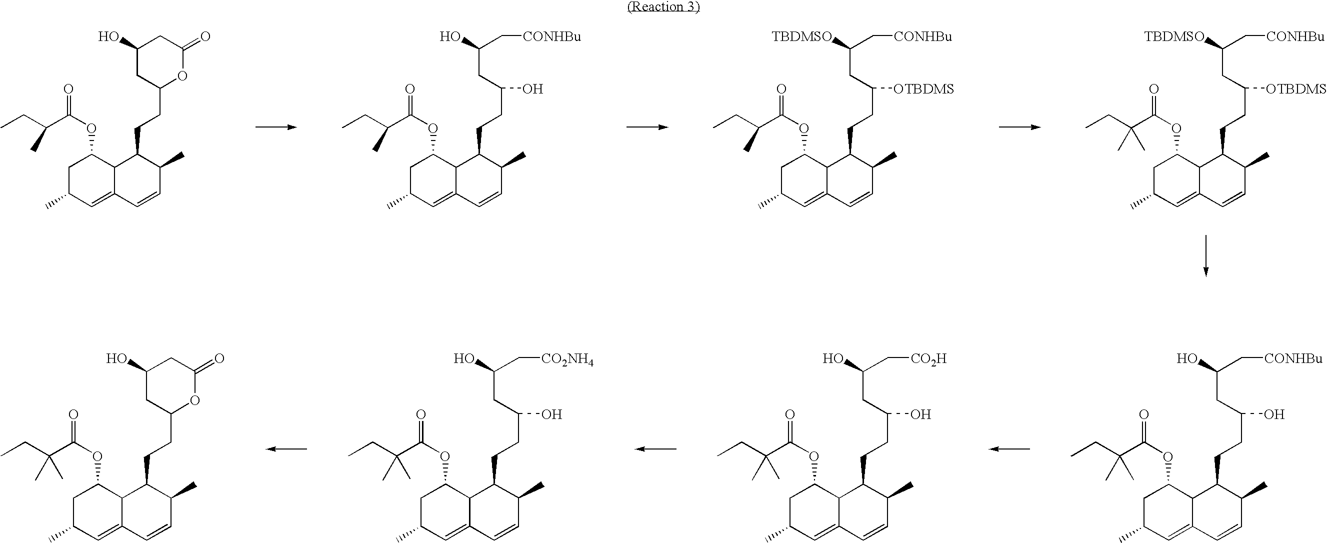

### SUMMARY OF THE INVENTION

The object of the invention is to provide a novel preparation method for making simvastatin by using inexpensive and easily available reagents under mild conditions with easy operation, to overcome the deficiencies in prior art.

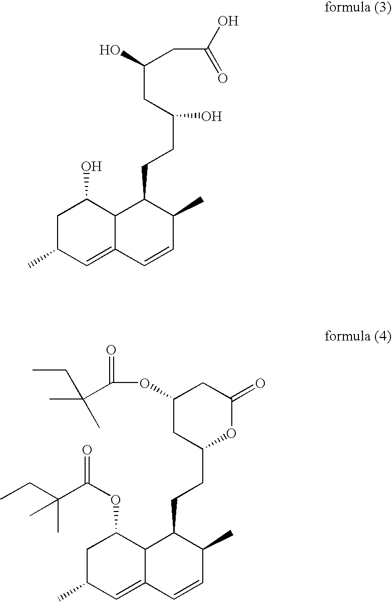

The preparation method of the present invention includes: firstly, hydrolyzing lovastatin by inorganic base(s) to form the compound of the formula (3), i.e., trihydroxy acid intermediate; secondly, directly esterifying the trihydroxy acid intermediate to prepare the compound of the formula (4), i.e., a simvastatin derivative of ([(1S,3R,7R,8S,8aR)-8-[2-[(2R,4R)-4-hydroxy-6-oxo-oxan-2-yl]ethyl]-3,7-dimethyl-1,2,3,7,8,8a-hexahydronaphthalen-1-yl]2,2-dimethylbutanoate), then carrying out a ring-opening reaction of cyclohexyl ester by catalysis, to form a ring-opened ester such as a methyl ester as shown in a formula (6), which is then catalyzed or acidified by methylamine or enzyme(s) to form simvastatin;

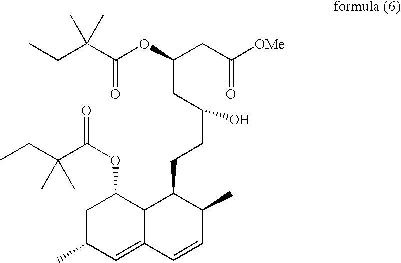

Alternatively, conditions of catalytic esterification in the present invention can be used to improve prior methods, such as, transferring the compound of the formula (3), i.e., trihydroxy acid intermediate into a compound of a formula (7), i.e., a six-membered ring ketal intermediate, then carrying out chemically catalytic esterification to form a compound of a formula (8), i.e., a simvastatin derivative, then providing simvastatin of the formula (1) via deprotection catalyzed by acid(s).

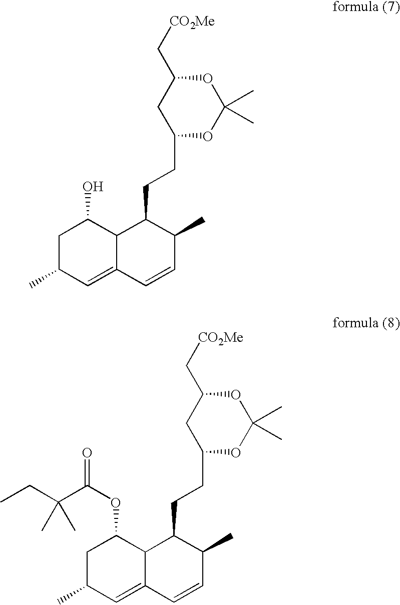

The present invention also relates to a separation method for the trihydroxy acid intermediate of the formula (3), which includes concentrating, adding ether, and acidification under low temperature.

The present invention also relates to the simvastatin derivative of the formula (4) and preparation thereof, which includes treating the trihydroxy acid intermediate of formula (3) with the acylating agent of 2,2-dimethyl butyryl chloride.

Still the present invention relates to the simvastatin derivative of the formula (4) and preparation thereof, which includes treating the trihydroxy acid intermediate of formula (3) with the acylating agent of 2,2-dimethyl butyryl anhydride.

The present invention also relates to a preparation method for the ring-opened ester of the formula (6), which includes catalysis reaction of the simvastatin derivative of the formula (4) in the presence of methanol or ethanol.

The present invention also relates to a preparation method for the six-membered ring ketal intermediate of the formula (7), which includes catalysis reaction of the trihydroxy acid intermediate of the formula (3).

The present invention also relates to the simvastatin derivative of the formula (8) and preparation methods thereof, which includes catalytically esterifying the six-membered ring ketal intermediate of the formula (7 ) by an acylating reagent of 2,2-dimethyl butyryl chloride.

The method(s) of the present invention leave out the protective and deprotective steps in prior methods, and the esterification condition(s)in 8-position greatly simplify the methods in prior art.

### DESCRIPTION OF THE INVENTION

After enormous experiments, the present inventor found that, under novel reaction condition, the compound of the formula (3) can be selectively transferred into the compound of the formula (4) or the compound of the formula (7), both of which could be handled in subsequent process to form simvastatin, thus the present invention is accomplished, and the abovementioned novel reaction condition is mild, and easy for operation. Furthermore, the novel and creative compounds of the formula (3), (4), (6) are provided during the reaction, presented here for the invention application.

The present invention is now illustrated in details. Firstly, lovastatin is hydrolyzed by the catalysis of the following inorganic base(s) to form the compound of the formula (3), i.e., trihydroxy acid intermediate (reaction 4).

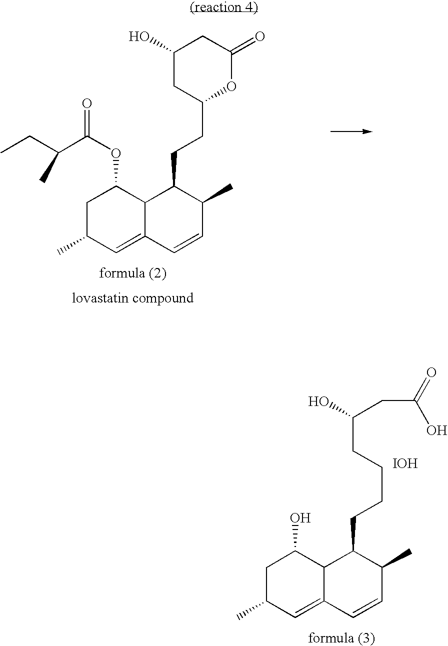

The inorganic base(s) may be potassium hydroxide, sodium hydroxide, lithium hydroxide, etc., the amount of the inorganic base may be 5 to 16 moles, generally 9 to 12 moles. This reaction can be carried out in the presence of pure water, or additional organic alcohols such as methanol, ethanol, propanol, isopropanol, butanol, isobutanol, tert-butanol, etc., which help to dissolve lovastatin, and proportion of the alcohol(s) to water may range from 1:1 to 10:1. It is advantageous to employ those organic alcohols that can form azeotrope with water, such as ethanol, propanol, isopropanol, butanol, isobutanol, tert-butanol, etc. The hydrolysis temperature may range from 30° C. to 100° C., and the hydrolysis duration may range from 6 to 48 hours. Concentration of lovastatin generally ranges from 1% to 12% (weight/volume ratio).

A post-treatment of the above step has great improvement compared with known methods, i.e., after the reaction, the resultant reaction solution is concentrated to one fifth to one tenth of its original volume, obtaining the concentrated solution, into which ether is added, with about one tenth to one half volume of the concentrated solution, then inorganic acid(s), such as hydrochloric acid, sulfuric acid, phosphoric acid, is used to neutralize and acidify under low temperature (0° C. to 10° C.), then the reaction product trihydroxy acid crystallizes and precipitates as solid in ether, thus simplifies the separation process. The condition of low temperature refers to 0° C. to 10° C.

Secondly, directly esterifies the compound of formula (3) to afford the compound of formula (4), i.e., the simvastatin derivative (reaction 5). This reaction does not require the protection and deprotection as in prior art.

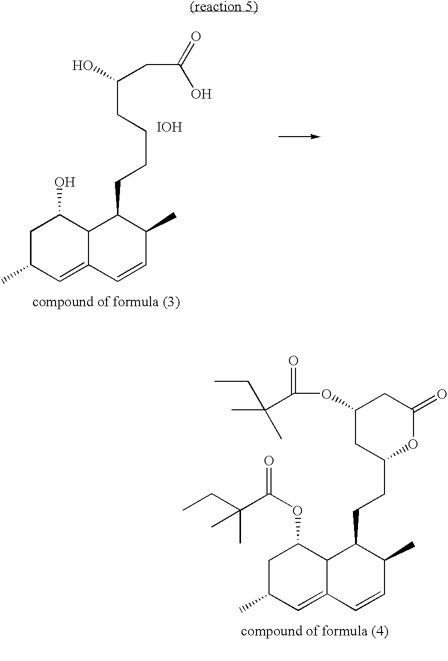

In this step of reaction, an intermediate of dihydroxy compound in a formula (5) is produced, however, the intermediate, which does not need to be separated, directly undertakes subsequent steps to yield the simvastatin derivative of the formula (4).

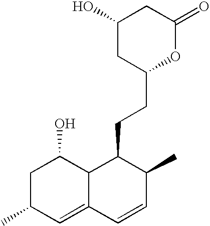

intermediate of dihydroxy compound of formula (5)

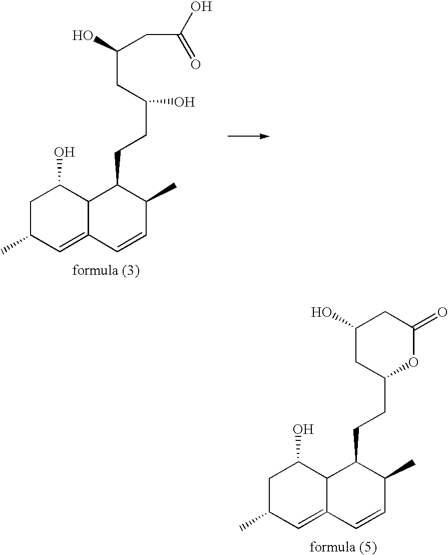

The compound of formula (3), trihydroxy intermediate, is treated by strong organic acid(s), such as para-toluenesulfonic acid, xylenesulfonic acid, benzenesulfonic acid, methanesulfonic acid, in the presence of an organic solvent(s) to convert into the dihydroxy intermediate of the formula (5). The organic solvent(s) employed may include but not limited to: dichloromethane, 1,2-dicholoroethane, toluene, hexane, ethyl acetate, isopropyl acetate, and acetonitrile.

Catalyst(s) employed in this reaction may be non-nucleophilic strong organic acid(s), such as para-toluenesulfonic acid, methanesulfonic acid, etc., or inorganic acid(s) such as sulfuric acid, phosphoric acid, etc., or acidic ion exchange resin(s), etc. When the starting material of the reaction is 1 mol, the amount of the catalyst(s) used in the reaction may range from 0.1 mol % to 100 mol %, generally from 1 mol % to 5 mol %.

Typically, this reaction is carried out under the protection of inert gas, such as nitrogen or helium, at a temperature from −20° C. to 60° C., mostly from 0° C. to 30° C.

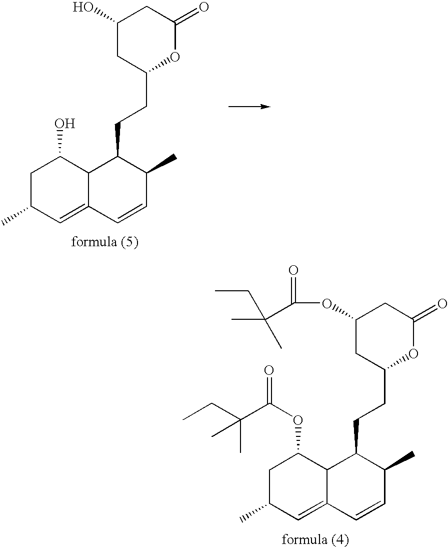

When it is determined by analysis that the conversion into the compound of formula (5) is almost complete, esterification reaction can be carried out to produce the simvastatin derivative of the formula (4). In this step of reaction, acylating reagent(s) are employed, such as 2,2-dimethyl butyryl chloride, to form the simvastatin derivative in the presence of organic solvent(s) with catalyst(s).

The above organic solvent(s) may be dichloromethane, 1,2-dichloroethane, toluene, or N,N-dimethyl formamide; the catalyst(s) may be quaternary ammonium halide(s), such as tetrabutylammonium chloride, tetrabutylammonium bromide, etc., or quaternary-phosphonium halide(s), such as tetraphenylphosphonium bromide, tetraphenylphosphonium iodide, tetrabutylphosphonium bromide, etc., the catalyst(s) may also be metallic halide(s), such as lithium bromide, zinc bromide, magnesium bromide, potassium bromide, lithium chloride, zinc chloride, magnesium chloride, nickel chloride, or iron chloride, etc. When the starting material of the reaction is 1 mol, the amount of the catalyst(s) used in the reaction may range from 0.2 mol to 3.0 mol, generally from 0.5 mol to 1.2 mol.

When 2,2-dimethyl butyryl chloride is used as acylating reagent, organic amine(s), such as triethylamine, N,N-diisopropyl ethylamine, or pyridine, may be added as trapping reagent for hydrochloride generated during reaction.

This step of reaction can also use 2,2-dimethyl butyryl anhydride as acylating reagent, to form the simvastatin derivative in the presence of organic solvent(s), such as dichloromethane, 1,2-dichloroethane, toluene, or N,N-dimethyl formamide, and by the action of catalyst(s). Generally, the catalyst(s) for esterification reaction may be Lewis acid(s), such as boron fluoride, iron trifluoromesylate, zinc fluoromesylate, copper fluoromesylate, ammonium fluoromesylate(s), or bismuth fluoromesylate. When the starting material of the reaction is 1 mol, the amount of the catalyst(s) used in the reaction may range from 0.01 mol to 2.0 mol, generally from 0.05 mol to 0.5 mol.

Typically, this step of reaction is carried out under the protection of inert gas, at a temperature from −20° C. to 60° C., mostly from 0° C. to 30° C.

Thirdly, the simvastatin derivative of the formula (4) is converted to compound of formula (6) through a ring-opening reaction of cyclohexyl ester in the presence of catalyst(s), the ring-opened esters of formula (6) include ring-opened methyl ester or ethyl ester (reaction 6).

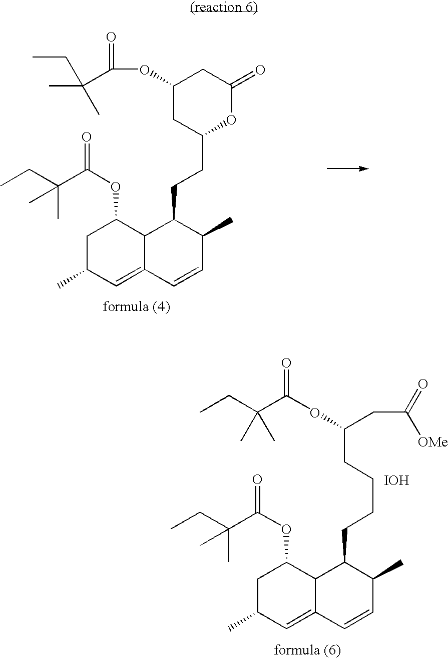

This step of reaction is generally carried out using methanol or ethanol as the solvent, producing corresponding methyl ester(s) (as shown in reaction 6) or ethyl ester(s). The catalyst(s) under such reaction condition may generally be quaternary ammonium halide(s), such as tetrabutylammonium chloride, tetrabutylammonium bisulfate, tetrabutylammonium bromide, etc., or quaternary phosphonium halide(s), such as tetraphenylphosphonium bromide, tetraphenylphosphonium iodide, tetrabutylphosphonium bromide, etc., and may also be Lewis acid(s), such as boron fluoride, iron trifluoromesylate, zinc fluoromesylate, copper fluoromesylate, ammonium fluoromesylate(s), or bismuth fluoromesylate. When the starting material of the reaction is 1 mol, the amount of the catalyst(s) used in the reaction may range from 0.1 mol to 2.0 mol, generally from 0.5 mol to 1.0 mol.

Typically, this step of reaction is carried out under the protection of inert gas, at a temperature from 20° C. to reflux temperature, mostly from 30° C. to 60° C.

Finally, treat the ring-opened ester of the formula (6) to prepare simvastatin (reaction 7):

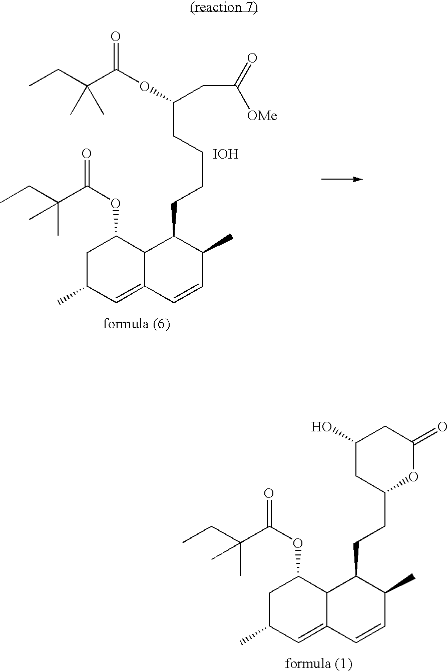

Treat the ring-opened methyl ester or ethyl ester obtained in the previous step with ammonia or aqueous solution of methylamine under a temperature from 0° C. to 30° C., to remove 2,2-dimethyl butyryl group in 4-position. This reaction condition will not affect the 2,2-dimethyl butyryl group in the 8-position. Ester(s) with ester group(s) at terminal of molecule, such as methyl ester shown in reaction 7, may be transferred into ammonium salt and amide in part. With the treatment of an acid(s), both methylammonium salt and formamide are totally transferred into simvastatin. Acid(s) used in the reaction may be hydrochloric acid, sulfuric acid, or all kinds of sulfonic acids.

This step of treatment may also include catalytic transformation by enzymes and subsequent conversion into simvastatin totally with the treatment of an acid(s). The enzyme catalyst(s) may be ester hydrolase(s), such as that abstracted from rabbit serum; and the acid(s) may be hydrochloric acid, sulfuric acid, or all kinds of sulfonic acids.

The above reaction condition(s) disclosed in the present invention for catalytic acylation using 2,2-dimethyl butyryl group in 8-position may also be used in known prior methods, which includes protecting the meta-dihydroxy groups in the trihydroxy acid. Compared with known preparation methods, the novel process avoids using anhydrous pyridine as a solvent, and substantially reduces the amount of 2,2-dimethyl butyryl chloride for esterification, for example, the required amount corresponding to 1 mole of starting material is cut from more than eight mole to 1.1 mole.

The following illustrate the improvement of the present invention to a known method.

The illustration below describes in details about another preparation method for simvastatin from the trihydroxy acid intermediate, which is prepared by the above-mentioned method.

In the first step, protection of the meta-dihydroxy groups and carboxyl group in the trihydroxy acid intermediate of the formula (3) forms a six-membered ring ketal intermediate of formula (7), i.e., transform the meta-dihydroxy groups in the trihydroxy acid intermediate of the formula (3) into a ketal with a six-membered ring, such as an acetal as shown in a following reaction, at the same time, transform the carboxyl group in the molecule to a corresponding ester, such as a methyl ester shown as in the following reaction (reaction 8).

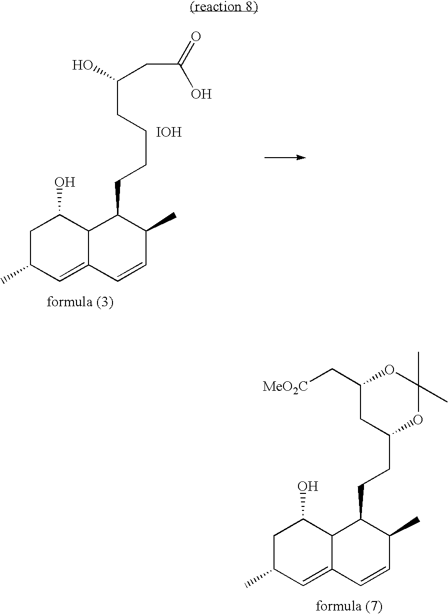

Suitable reagent(s) for this step may be corresponding ketone(s), or corresponding 2,2-dialkoxypropane. The alkoxy group in the 2,2-dialkoxypropane may be of C1˜C3 alkoxy, such as 2,2-dimethoxypropane, 2,2-diethoxypropane, 2-methoxy-2-ethoxypropane or 2,2-dipropoxypropane, etc. Corresponding ketone may be employed as starting material, such as acetone, methyl ethyl ketone, or pentanone, etc, meanwhile the desirable corresponding alcohol(s) may be methanol, ethanol, or propanol, etc.

Catalyst(s) employed in the reaction may be non-nucleophilic strong organic acid(s), such as para-toluenesulfonic acid, methanesulfonic acid, etc., or inorganic acid(s) such as sulfuric acid, phosphoric acid, etc., or acidic ion exchange resin(s), etc.

Corresponding reagent(s) may be directly employed in this reaction as reaction solvent, such as 2,2-dimethoxypropane, etc., and organic substance(s), such as toluene, dichloromethane, dichloroethane, etc., may be also used as reaction solvent. Under such condition, the ratio of the reagent and the starting material, i.e. dihydroxy intermediate of formula (5) that is an lactone, is generally from 1:1 to 2:1.

Typically, this step of reaction is carried out under the protection of inert gas, at a temperature from −20° C. to 60° C., mostly from 0° C. to 30° C. When the starting material of the reaction is 1 mol, the amount of the catalyst(s) used in the reaction may range from 0.1 mol % to 100 mol %, generally from 1 mol % to 5 mol %.

In the second step, catalytic esterification of the six-membered cycloketal intermediate of the formula (7) is carried out (reaction 9):

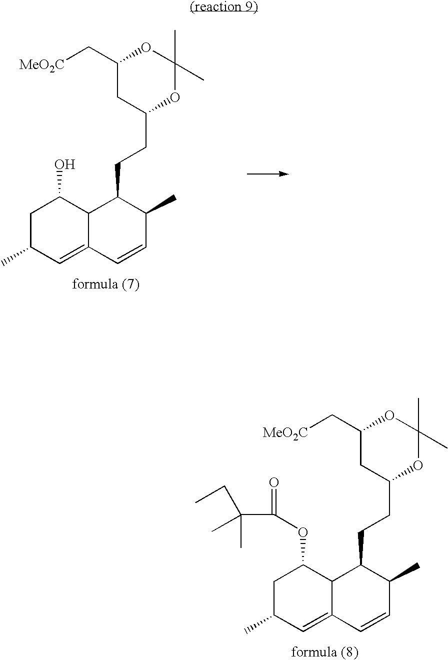

This step of reaction is carried out in an organic solvent, such as dichloromethane, 1,2-dichloroethane, toluene, or N,N-dimethyl formamide, using 2,2-dimethyl butyryl chloride as acylating reagent(s), in the presence of catalyst(s), to form the simvastatin derivative of the formula (8).

Under the reaction conditions, the esterification catalyst(s) may be quaternary ammonium halide(s), such as tetrabutylammonium chloride, tetrabutylammonium bromide, etc., or quaternary-phosphonium halide(s), such as tetraphenylphosphonium bromide, tetraphenylphosphonium iodide, tetrabutylphosphonium bromide, etc.; the catalyst(s) may also be metallic halide(s), such as lithium bromide, zinc bromide, magnesium bromide, potassium bromide, lithium chloride, zinc chloride, magnesium chloride, nickel chloride, or iron chloride, etc. When the starting material of the reaction is 1 mol, the catalyst(s) used in the reaction may range from 0.2 mol to 3.0 mol, generally from 0.5 mol to 1.2 mol.

When 2,2-dimethyl butyryl chloride is used as acylating reagent, organic amine(s), such as triethylamine, N,N-diisopropyl ethylamine, or pyridine, may be added as trapping reagent for hydrochloride generated during reaction. Typically, this step of reaction is carried out under the protection of inert gas, at a temperature from 20° C. to 60° C., mostly from 30° C. to 50° C.

In the third step, deprotection reaction is carried out by acid catalysis, to form simvastatin (reaction 10).

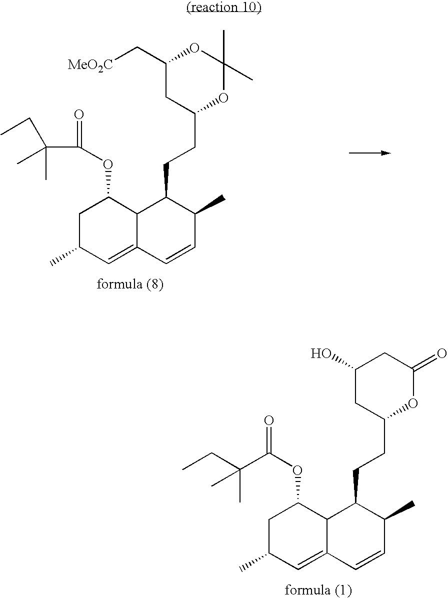

Typically, the reaction is carried out in a multi-component solvent, which is a mixture of water and another component such as acetonitrile, tetrahydrofuran, toluene, or N,N-dimethyl formamide. Acid(s) used in the reaction may be hydrochloric acid, sulfuric acid, or all kinds of sulfonic acids. The reaction is carried out at a temperature from 0° C. to 60° C., mostly from 0° C. to 20° C., under the protection of inert gas.

Addition of hexane or heptane into the toluene solution of the raw product can promote crystallization, precipitation and isolation of simvastatin.

The followings illustrate the intermediates and the methods of the present invention, in combination of the below examples. It is understood that these examples are illustrative and are not to be read as limiting the scope of the invention as it is defined by the claims. If any experiment methods in the following examples have not been specified by detailed experiment conditions, it should conform to normal conditions, or those conditions suggested by manufacture(s). Proportion and percentage are based on mole, unless specified otherwise.

### EXAMPLE 1

### Hydrolysis of Lovastatin And Formation of Trihydroxy Acid Intermediate (Reaction 4)

Under the protection of nitrogen, 20.0 gram of lovastatin was dissolved in 200 ml of heated ethanol. At room temperature, 100 ml of cooled aqueous solution of potassium hydroxide (36 g) was slowly added into the above reaction solution. The reaction mixture was stirred for 0.5 to 1 hour under the protection of nitrogen in room temperature, and then refluxed for 12 to 16 hours. Additionally, added 300 ml of water, vaporized to remove 500 ml of solvent, then cooled to a temperature of 5˜10° C. Then added 80 ml of ether, and adjusted pH value to 5.0 by slowly adding concentrated hydrochloric acid, meanwhile controlling temperature within a range from 5 to 10° C. Stirred for another 1 hour, then the trihydroxy acid intermediate in ether crystallized and precipitated, and the solid product obtained was washed with water and dried in vacuum, weighed 15.6 gram (yield: 93%).

Melting point (m.p.): 127˜129° C.

¹H-NMR (δ, CDCl₃): 5.96 (d, 1H), 5.79 (dd, 1H), 5.54 (br, 1H), 4.33 (m, 1H), 4.28 (m, 1H), 3.96 (m, 1H), 2.2-2.6 (m, 7H), 1.1-1.9 (m, 10H), 1.18 (d, 3H), 0.91 (d, 3H).

### EXAMPLE 2

### Synthesis of the Simvastatin Derivative of the Formula (4) (Reaction 5)

Under the protection of nitrogen, 16.0 gram of dried trihydroxy acid intermediate was suspended in 300 ml of dichloromethane. After the addition of 0.4 gram of para-toluenesulfonic acid, the reaction mixture is heated to reflux and vaporized about 100 ml of dichloromethane. The white solid soon disappeared and dissolved to provide a transparent solution. Then cooled down to a temperature of 5 to 10° C., subsequently added 0.5 mol of lithium bromide, 2.1 mol of triethylamine, 2.4 mol of 2,2-dimethyl-butyryl chloride. The reaction mixture was stirred for 0.5 to 1 hour under the protection of nitrogen, then stirring was continued at room temperature. Once the reaction is completed, 100 ml water was added, stirred for 30 minutes to separate its organic phase. The organic phase was washed with saturated salt solution for once (100 ml), saturated sodium bicarbonate solution (aqueous) for four times (100 ml per time), and saturated salt solution for twice (100 ml per time), then it was dried with sodium sulfate, filtered, and vaporized to get rid of the solvent, to produce the simvastatin derivative of the formula (4), weighing 22.1 gram (yield 90.6%).

Melting point (m.p.) 6.2 to 6.6° C.

¹H-NMR (δ, CDCl₃): 5.93 (d, 1H), 5.71 (dd, 1H), 5.44 (br, 1H), 5.29 (m, 1H), 5.18 (m,1H), 4.38 (m, 1H), 2.68 (m, 3H), 2.17-2.41 (m, 4H), 1.32-1.98 (m, 11H), 1.09 (br, 12H), 1.06 (d, 3H), 0.84 (d, 3H), 0.78 (m, 6H).

### EXAMPLE 3

### The Ring-Opening Reaction of Cyclohexyl Ester of the Simvastatin Derivative of the Formula (4) (Reaction 6)

Under the protection of nitrogen, 12.0 gram of dried simvastatin derivative of the formula (4) is dissolved in 200 ml of methanol. Added 0.5 mol of tetrabutylammonium bisulfate, then heated the reaction mixture and fluxed until 95% of the simvastatin derivative of the formula (4) was converted to corresponding ring-opened cyclohexyl ester compound. Vaporize to remove all the solvent, and the raw product obtained was stirred in 200 ml of water and 200 ml of heptane for 2 hours. The organic phase isolated was dried with sodium sulfate, filtered, then vaporized to get rid of solvent to obtain the target product, weighing 11.2 gram (yield 88%)

Melting point (m.p.) 3.3 to 3.6° C.

¹H-NMR (δ, CDCl₃): 5.93 (d, 1H), 5.71 (dd, 1H), 5.44 (br, 1H), 5.29 (m, 1H), 5.18 (m,1H), 4.38 (m, 1H), 3.61 (s, 3H), 2.68 (m, 4H), 2.17-2.41 (m, 4H), 1.32-1.98(m, 11H), 1.09 (br, 12H), 1.06 (d, 3H), 0.84 (d, 3H), 0.78 (m, 6H).

### EXAMPLE 4

### Synthesis of Simvastatin (Reaction 7)

Under the protection of nitrogen, 12.0 gram of dried ring-opened methyl ester is dissolved in 100 ml of acetonitrile. The solution was cooled to a temperature of 5˜10° C., then 10 mol of aqueous methylamine is added. The reaction mixture was stirred for 0.5 to 1 hour under the protection of nitrogen, and then it was stirred continuously at room temperature. After all the starting materials disappeared, it was cooled to 0˜5° C. again. Added concentrated hydrochloric acid to neutralize excessive methylamine, then continued to add concentrated hydrochloric acid until the concentration of the hydrochloric acid in the reaction liquid reached 4M. Stirring is continued over night at a temperature of 0 to 5° C., then simvastatin is extracted with 500 ml of toluene. The organic phase isolated was washed with water for once (100 ml), saturated sodium bicarbonate solution (aqueous) for twice (100 ml per time), and saturated salt solution foe twice (100 ml per time), then it was dried with sodium sulfate. Filtered, and concentrated to a volume of 80 ml, then added triple volume of hexane. Slowly stirred over night at a temperature of 5° C., then filtered to produce simvastatin, weighing 6.5 gram(yield 71%).

Melting point (m.p.): 133-135° C.

¹H-NMR (δ, CDCl₃): 6.01 (d, 1H), 5.78 (dd, 1H), 5.51 (br, 1H), 5.37 (m, 1H), 4.62 (m,1H), 4.39 (br, 1H), 2.73-2.63 (m, 2H), 2.22-2.48 (m, 4H), 1.32-1.98 (m, 11H), 1.14 (s, 3H), 1.13 (s, 3H), 1.09 (d, 3H), 0.89 (d, 3H), 0.82 (t, 3H).

### EXAMPLE 5

### Synthesis of Six-Membered Ketal (Reaction 8)

Under the protection of nitrogen, 12.0 gram of dried trihydroxy acid intermediate was suspended in 300 ml of dichloromethane. After 0.4 gram of para-toluenesulfonic acid is added, the reaction mixture is heated to reflux and vaporized to remove about 100 ml of dichloromethane. The white solid soon disappeared and dissolved to provide a transparent solution. Then cooled to a temperature of 5 to 10° C. Added 10 mol of 2,2-dimethoxypropane, and continued stirring for another 1 hour at room temperature, then 3 gram of sodium bicarbonate is added, and continued stirring for 30 minutes. The reaction solution was washed with water for once (100 ml), saturated sodium bicarbonate solution (aqueous) for once (100 ml), and saturated salt solution for once (100 ml), then it was dried with sodium sulfate, filtered, and vaporized to get rid of the solvent, to produce the target product, weighing 13.36 gram (yield 96%).

Melting point (m.p.) 3.1 to 3.5° C.

¹H-NMR (δ, CDCl₃): 5.99 (d, 1H), 5.78 (dd, 1H), 5.54 (br, 1H), 4.33 (m, 1H), 4.28 (m, 1H), 3.85 (br, 1H), 3.65 (s, 3H), 2.2-2.6 (m, 5H), 1.1-1.9 (m, 10H), 1.43 (s, 3H), 1.38 (s, 3H), 1.18 (d, 3H), 0.90 (d, 3H).

### EXAMPLE 6

### Chemical Catalytic Esterification (Reaction 9), i.e. “Synthesis of the Simvastatin Derivative of the Formula (8)”

Under the protection of nitrogen, 10.0 gram of dried six-membered ring ketal intermediate is dissolved in 100 ml of dichloromethane. Cooled the solution to a temperature of 5 to 10° C., then 0.5 mole of lithium bromide is added, followed by 1.1 mol of pyridine, and 1.2 mol of 2,2-dimethyl-butyryl chloride. The reaction mixture was stirred for 0.5 to 1 hour under the protection of nitrogen, then heated to reflux and continued stirring. After the reaction is completed, 100 ml water is added to terminate the reaction, then stirred for 30 minutes to separate its organic phase. The isolated organic phase was washed with saturated salt water for once (100 ml), saturated sodium bicarbonate solution (aqueous) for three times (100 ml per time), and saturated salt water for twice (100 ml per time), then it was dried with sodium sulfate, filtered, and vaporized to get rid of the solvent, to produce the simvastatin derivative of the formula (8), weighing 11.37 gram (yield 91%)

Melting point (m.p.) 4.7 to 5.1° C.

¹H-NMR (δ, CDCl₃): 5.99 (d, 1H), 5.78 (dd, 1H), 5.54 (br, 1H), 5.33 (m, 1H), 4.29 (m, 1H), 3.71 (br, 1H), 3.65 (s, 3H), 1.8-2.6 (m, 5H), 1.45 (s, 3H), 1.35 (s, 3H), 1.1-1.7 (m, 11H), 1.12 (s, 3H), 1.11 (s, 3H), 1.08 (d, 3H), 0.90 (d, 3H), 0.88 (t, 3H).

### EXAMPLE 7

### Deprotection Reaction By Acid Catalysis, i.e., Synthesis of Simvastatin (Reaction 10)

Under the protection of nitrogen, 10.0 gram of dried simvastatin derivative of the formula (8) was dissolved in 100 ml of acetonitrile. At a temperature of 0 to 5° C., 100 ml of 4M hydrochloric acid is added, stirred continuously over night, then extracted simvastatin with 500 ml of toluene. The isolated organic phase was washed with water for once (100 ml), saturated sodium bicarbonate solution (aqueous) for twice (100 ml per time), and saturated salt water for twice (100 ml per time), then it was dried with sodium sulfate, filtered, and concentrated to a volume of 80 ml, then added triple volume of hexane. Slowly stirred over night at the temperature of 5° C., then filtered to produce simvastatin, weighing 7.16 gram (yield 84%).

Melting point (m.p.) 133 to 135° C.

¹H-NMR (δ, CDCl₃): 6.01 (d, 1H), 5.78 (dd, 1H), 5.51 (br, 1H), 5.37 (m, 1H), 4.62 (m,1H), 4.39 (br, 1H), 2.73-2.63 (m, 2H), 2.22-2.48 (m, 4H), 1.32-1.98 (m, 11H), 1.14 (s, 3H), 1.13 (s, 3H), 1.09 (d, 3H), 0.89 (d, 3H), 0.82 (t, 3H).

All the documents cited herein are incorporated into the invention as reference, as if each of them is individually incorporated. Further, it would be appreciated that, in light of the above described teaching of the invention, the skilled in the art could make various changes or modifications to the invention, and these equivalents would still be within the scope of the invention defined by the appended claims of the application.

1-9. (canceled)

10. A simvastatin derivative of formula (4), which has the following structure:

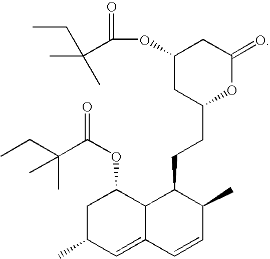

11. A simvastatin derivative of formula (6), which has the following structure:

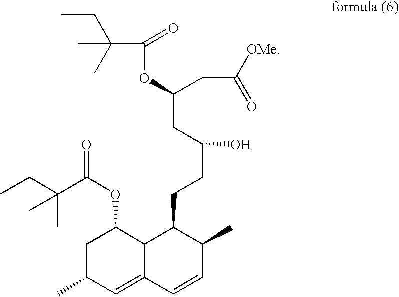

12. An isolation method of the compound of formula (3), which has the following structure: 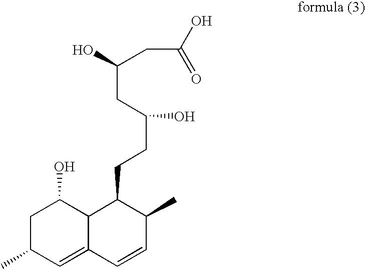
comprising the steps of:
(i) concentrating a reaction solution containing the compound of formula (3), until the volume thereof is one fifth to one tenth of its original volume, obtaining a concentrated solution;
(ii) adding ether, with its volume being one tenth to one half of said concentrated solution;
(iii) neutralizing and acidifying with inorganic acid at a low temperature, forming said compound of formula (3), wherein the low temperature ranges from 0° C. to 10° C.

13. A synthesis method of the compound of formula (4) according to claim 1, comprising the steps of:
(i) preparing and isolating the compound of formula (3); 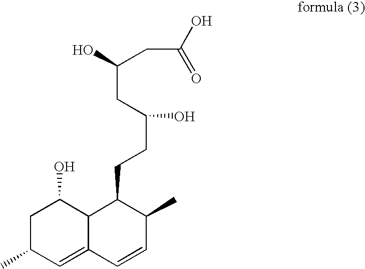
(ii) treating said compound of formula (3) with a acylating reagent, forming a compound of formula (4), wherein said acylating reagent is selected from the group consisting of 2,2-dimethyl butyryl chloride or 2,2-dimethyl butyryl anhydride.

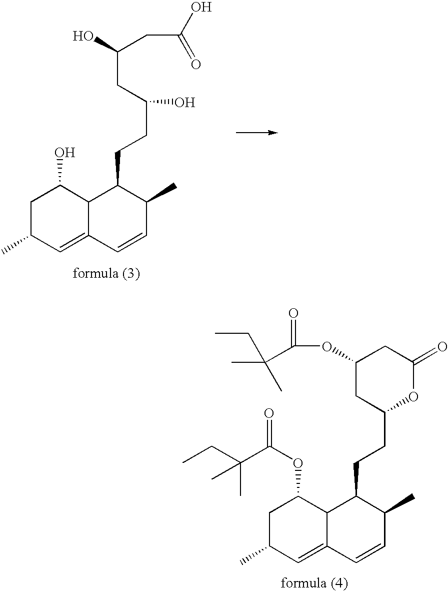

14. A synthesis method of the compound of formula (6), 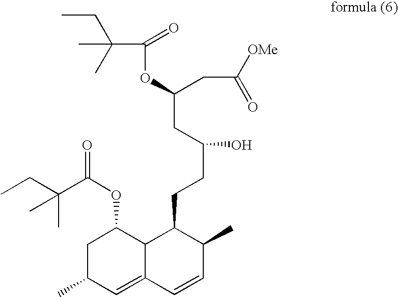
comprising the steps of:
(i) preparing and isolating the compound of formula (3); 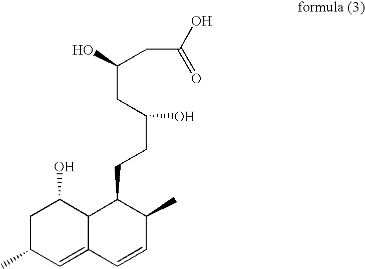
(ii) treating said compound of formula (3) with an acylating reagent, to obtain said compound of formula (4), 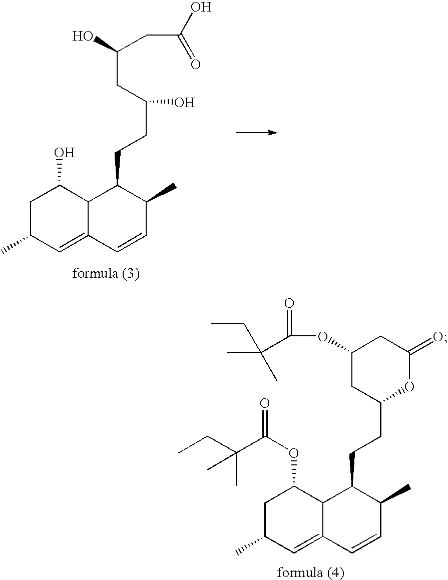
wherein said acylating reagent is selected from the group consisting of 2,2-dimethyl-butyryl chloride and 2,2-dimethyl butyryl anhydride;
(iii) catalyzing the compound of formula (4) in the presence of a polar solvent, to form the compound of formula(6), 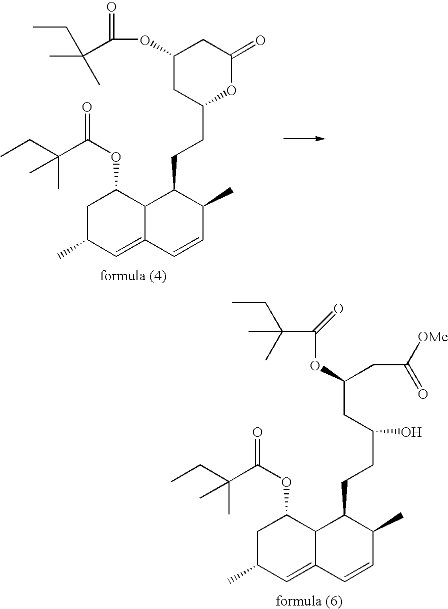
wherein said polar solvent is one or more selected from the group consisting of methanol and ethanol.

15. A synthesis method of the simvastatin derivative of formula (8), 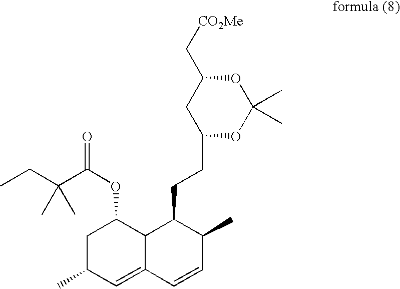
comprising the steps of:
(i) converting the compound of formula (3) to a compound of formula (7); 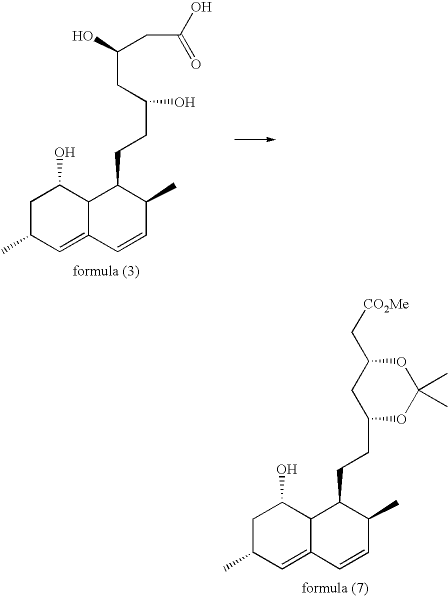
(ii) esterifying the compound of formula (7) with an acylating reagent, to obtain the compound of formula (8), wherein said acylating reagent is one or more selected from the group consisting of 2,2-dimethyl butyryl chloride and 2,2-dimethyl butyryl anhydride.

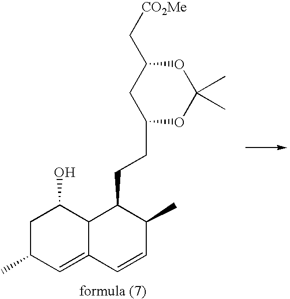

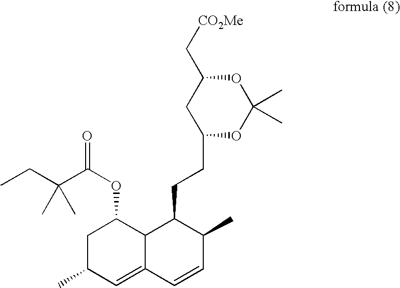

16. A synthesis method of the simvastatin of formula (1), comprising the steps of: 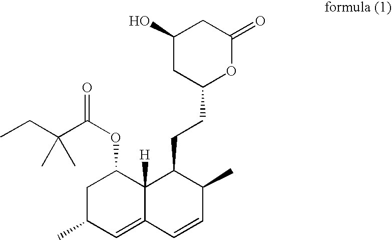
(i) hydrolyzing lovastatin of formula (2) with an inorganic base, to obtain the compound of formula (3); 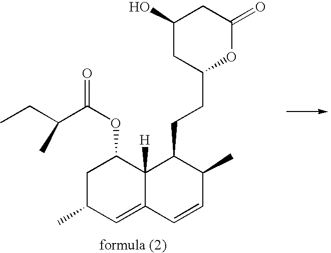 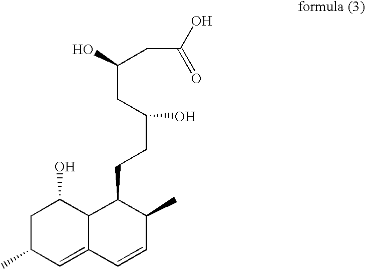
(ii) treating said compound of formula (3) with an acylating reagent in the presence of a catalyst in an organic solvent, to obtain the compound of formula (4); 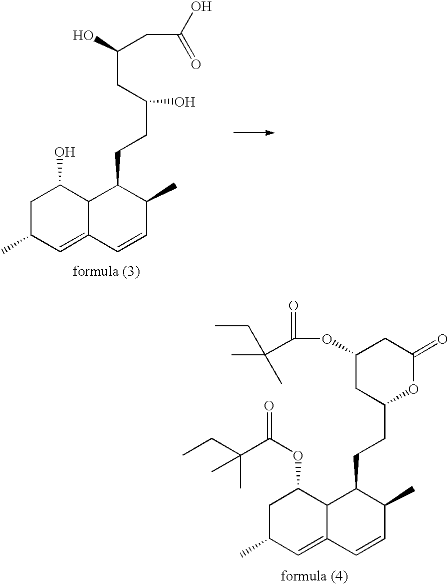
wherein said acylating reagent is one or more selected from the group consisting of 2,2-dimethyl butyryl chloride and 2,2-dimethyl butyryl anhydride; said organic solvent is one or more selected from the group consisting of dichloromethane, 1,2-dichloroethane, toluene, and N,N-dimethyl formamide; said catalyst is one or more selected from the group consisting of a quaternary ammonium halide, a quaternary phosphonium halide and a metallic halide;
(iii) treating the compound of formula (4) in the presence of a catalyst, to obtain the compound of formula (6), 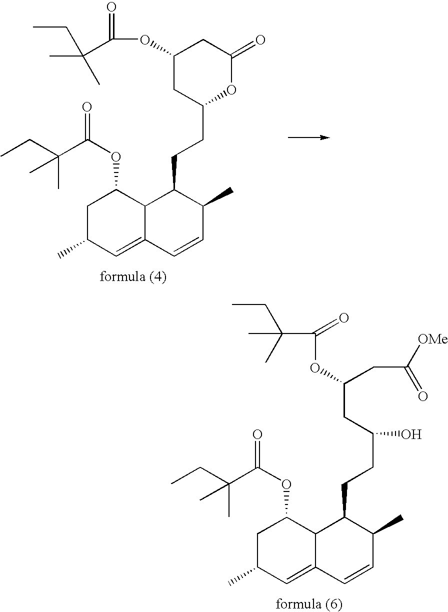
wherein said catalyst is selected from the group consisting of a quaternary ammonium halide, a quaternary phosphonium halide, or a Lewis acid;
(iv)catalyzing or acidizing the compound of formula (6) by methylamine or enzyme, to obtain simvastatin of formula (1);

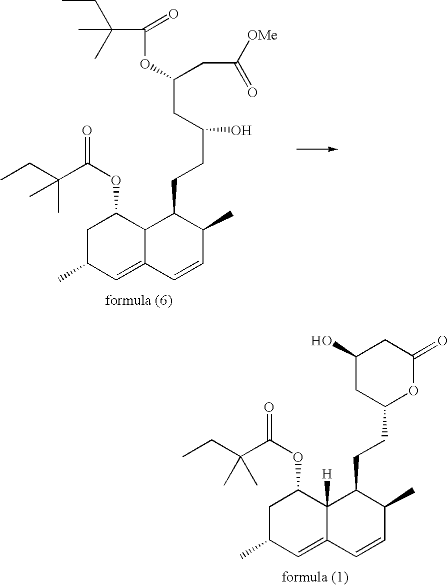

17. A synthesis method for simvastatin of formula (1), comprising the steps of: 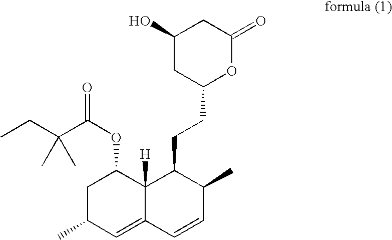
(i) treating the compound of formula (3) in the presence of a catalyst, to obtain the compound of formula (7), 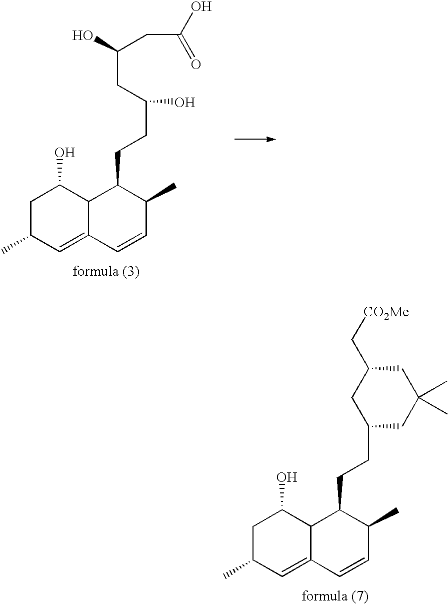
wherein said catalyst is selected from the group consisting of non-nucleophilic strong organic acid(s), inorganic acid(s), or acidic ion exchange resin(s);
(ii) treating the compound of formula (7) with an acylating reagent in the presence of a catalyst and a solvent, to obtain the simvastatin derivative of formula (8), 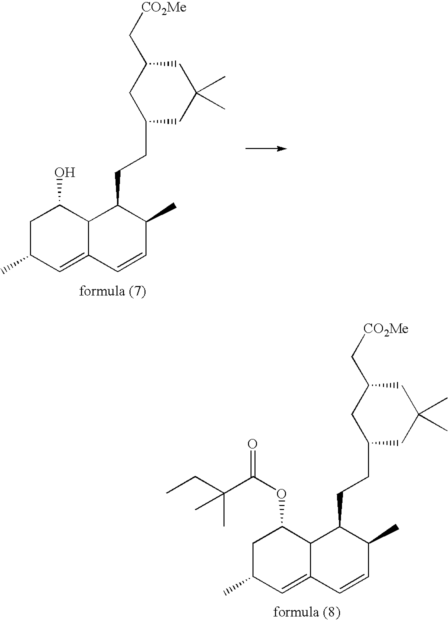
wherein said acylating reagent is one or more selected from the group consisting of 2,2-dimethyl butyryl chloride and 2,2-dimethyl butyryl anhydride;
said organic solvent is one or more selected from the group consisting of dichloromethane, 1,2-dichloroethane, toluene, and N,N-dimethyl formamide;
said catalyst is one or more selected from the group consisting of a quaternary ammonium halide, a quaternary phosphonium halide and a metallic halide;
(iii) deprotecting the compound of formula (8) by acid catalysis, to obtain simvastatin of formula (1). 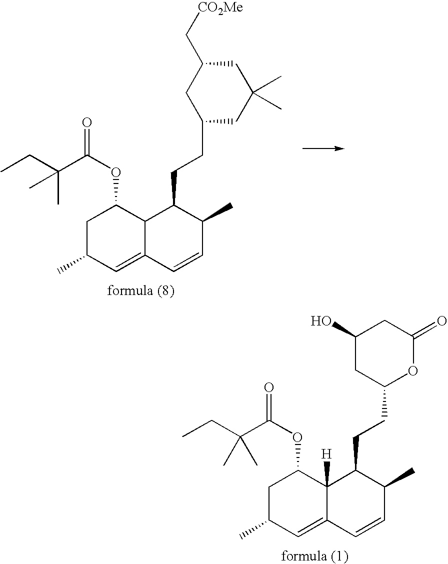
wherein said acid is selected from the group consisting of hydrochloric acid, sulfuric acid, sulfonic acid and combination thereof.

18. The use of the compound of formula (4) according to claim 1 in the manufacture of simvastatin for inhibiting hydroxylmethyl glutaryl coenzyme A reductase.
